(19) United States
(12) Patent Application Publication
(10) Pub. No.: US 2020/0215316 A1
(43) Pub. Date: Jul. 9, 2020

(54) Apparatus with Contrast-Coated Balloon and Methods

(71) Applicant: Konstantinos Marmagkiolis, Wesley Chapel, FL (US)

(72) Inventor: Konstantinos Marmagkiolis, Wesley Chapel, FL (US)

(21) Appl. No.: 16/239,250

(22) Filed: Jan. 3, 2019

(51) Int. Cl. A61M 31/00; A61K 49/04; A61L 29/18

(52) U.S. Cl. CPC A61M 31/005; A61M 2025/1031; A61L 29/18; A61K 49/0438

(57) ABSTRACT

An apparatus for treating a cardiac structural and vascular condition. The apparatus includes a catheter and a balloon disposed on the catheter. The balloon further includes a contrast medium covering the outer surface of the balloon, A method for treating a patient. A method for making a balloon with a contrast medium on an outer surface of the balloon.

### FIELD OF THE INVENTION

The present invention is related to medical implant devices, particularly, a contrast coated balloon and making and using the same. (As used herein, references to the “present invention” or “invention” relate to exemplary embodiments and not necessarily to every embodiment encompassed by the appended claims.)

### BACKGROUND OF THE INVENTION

This section is intended to introduce the reader to various aspects of the art that may be related to various aspects of the present invention. The following discussion is intended to provide information to facilitate a better understanding of the present invention. Accordingly, it should be understood that statements in the following discussion are to be read in this light, and not as admissions of prior art.

Balloons are generally cylindrical shaped devices that are radially expandable to open a segment of blood vessel or other anatomical structure (i.e. cardiac structures). Moreover, balloons are used to estimate the size of a vascular or other anatomical structure (i.e. aortic valve, patent foramen, pulmonic artery etc. More recently, balloons have been developed with coatings to deliver drugs or other therapeutic agents for therapeutic purposes.

Balloons are used in a variety of medical therapeutic applications including intravascular angioplasty. For example, a balloon catheter device (non-compliant, semi-compliant or compliant) is inflated during PTCA (percutaneous transluminal coronary angioplasty) to dilate a narrow blood vessel. The narrowing may be the result of a lesion such as a plaque or thrombus or from natural anatomic narrowings. After inflation, the pressurized balloon exerts a compressive force on the lesion thereby increasing the inner diameter of the a fleeted vessel. The increased interior vessel diameter facilitates improved blood flow.

When used to estimate the size of a vessel or other anatomic structure, the balloon (compliant or ultra-compliant) is inflated, allowing operators to take measurement based on the radio-opaque contrast inside the balloon while the balloon is inflated. Those balloons evaluate the shape and size of various anatomic structures like cardiac valves, atrial septal defect, patent foramen ovale, left atrial appendage etc. Traditionally this procedure takes place before placement of another cardiac or vascular device (percutaneous cardiac valve procedure, ASD or PFO closure device, PFO device, or intravascular stent). After inflation, the pressurized compliant balloon, which is filled with contrast, conforms inside the anatomic structure. Using angiography, the shape and size of the anatomic structure can be accurately estimated. This information is used to guide the size of the used device and the accurate location. However, after the deflation of the balloon, exact location of the anatomic structure can only be vaguely estimated as cardiac and vascular structures constantly move.

Balloons have been used with coatings to deliver drug or other therapy to the patient at the site of the stent, such as the interior wall of an artery or vessel. The coating is typically applied to the balloon by dipping or spraying the balloon with a liquid containing the drug or therapeutic agent dispersed in a polymer/Solvent mixture. The liquid coating then dries to a solid uniform coating. Combinations of dipping and Spraying can also be used. The dried coating forms a uniform radial layer over the balloon.

Although structure visualization is easy during balloon inflation when contrast is inside the balloon, consequent interventional procedures are problematic for the operator. Movement of the patient and continuous movement of cardiac structures alter the exact location of the structure of interest and important information derived during balloon inflation about location and size may change.

It would be desirable, therefore, to have the anatomic structures “marked” with contrast during balloon inflation and this “mark” remains during the subsequent procedures so the exact location of the structure of interest in the patient can be identified.

### BRIEF SUMMARY OF THE INVENTION

The present invention pertains to an apparatus for treating a patient. The apparatus comprises a catheter. The apparatus comprises a balloon having an outer surface disposed on the catheter. The balloon includes a contrast medium disposed on and covering the outer surface of the balloon. The balloon and catheter are adapted to be positioned in the patient. The balloon has an inflated state where the balloon is expanded in size and an uninflated state, where the balloon is unexpanded.

The present invention pertains to a method of making a balloon with a contrast medium of an outer surface of the balloon. The method comprises the steps of inflating the balloon. There is the step of placing the contrast medium on the outer surface of the balloon. There is the step of drying the contrast medium of the outer surface of the balloon. There is the step of deflating the balloon.

The present invention pertains to a method for treating a patient. The method comprises the steps of inserting a deflated balloon having an outer surface coated with a contrast medium with a catheter in the patient. There is the step of positioning the deflated balloon with the catheter to a desired position inside a vein or artery of the patient. There is the step of inflating the balloon so the balloon contacts and presses against an interior wall of the vein or artery or other anatomic cardiovascular structures of the patient. There is the step of depositing the contrast medium coating the outer surface of the balloon on the interior wall of the vein or artery or other anatomic cardiovascular structures. There is the step of deflating the balloon. There is die step of removing the balloon with the catheter from the patient. There is the step of positioning properly an object in the patient at the desired location by comparing a position of the object with the contrast medium deposited on the interior wall of the vein or artery or other anatomic cardiovascular structures.

### BRIEF DESCRIPTION OF THE SEVERAL VIEWS OF THE DRAWINGS

In the accompanying drawings, the preferred embodiment of the invention and preferred methods of practicing the invention are illustrated in which:

FIG. 1 is a schematic representation of a balloon with contrast medium on an outer surface of the balloon attached to a catheter of the present invention.

FIG. 2 is a cross-sectional view of the balloon of the present invention.

FIG. 3 shows the balloon in the inflated stale in a patient structure.

FIG. 4 shows the contrast medium remaining on the patient structure after the balloon has been removed.

### DETAILED DESCRIPTION OF THE INVENTION

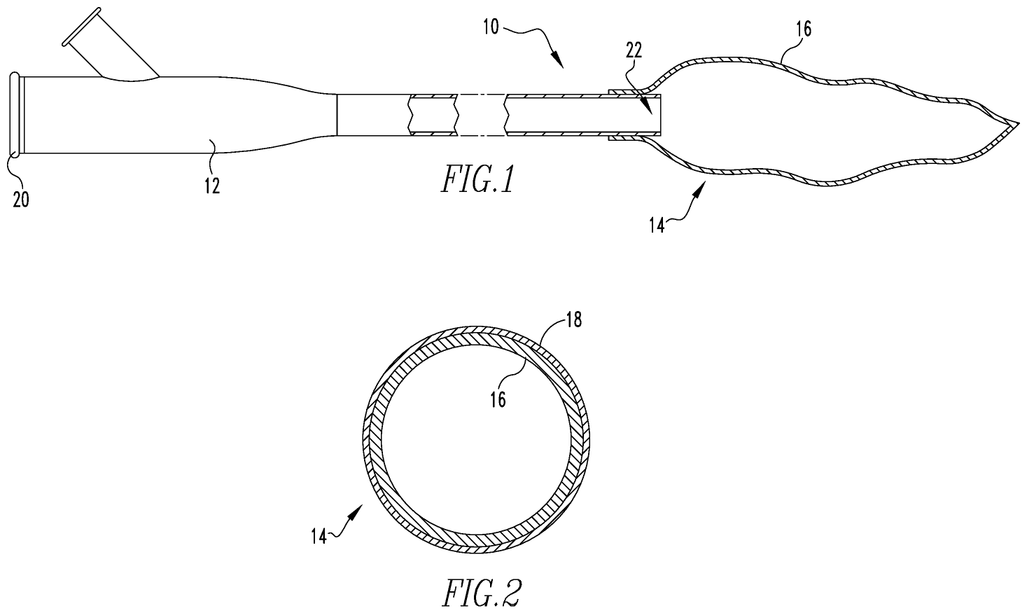

Referring now to the drawings wherein like reference numerals refer to similar or identical parts throughout the several views, and more specifically to FIGS. 1 and 2 thereof, there is shown an apparatus 10 for treating a patient. The apparatus 10 comprises a catheter 12. The apparatus 10 comprises a balloon 14 having an outer surface 16 disposed on the catheter 12. The balloon 14 includes a contrast medium 18 disposed on and covering the outer surface 16 of the balloon 14. The balloon 14 and catheter 12 are adapted to be positioned in the patient. The balloon 14 has an inflated state where the balloon 14 is expanded in size and an uninflated state, where the balloon 14 is unexpanded.

The contrast medium 18 may include either Iopamidol, Iohexol, ioversol or ioxaglate. The balloon 14 may have a diameter size ranging from 3 mm to 40 mm and a length from 15 mm to 80 mm. The catheter 12 may be hollow and has an input port 20 through which pressurized fluid flows to the balloon 14, and an output port 22 on which the balloon 14 is disposed to receive the pressurized fluid to inflate the balloon 14 into the inflated state. The pressurized fluid may also be a contrast medium 18 that includes either Iopamidol, Iohexol, ioversol or ioxaglate.

The present invention pertains to a method of making a balloon 14 with a contrast medium 18 of an outer surface 16 of the balloon 14. The method comprises the steps of inflating the balloon 14. There is the step of placing the contrast medium 18 on the outer surface 16 of the balloon 14. There is the step of drying the contrast medium 18 of the outer surface 16 of the balloon 14. There is the step of deflating the balloon 14.

The placing step may include the steps of rotating the balloon 14 and spraying the contrast medium 18 onto the outer surface 16 of the balloon 14. There may be the step of clamping the balloon 14.

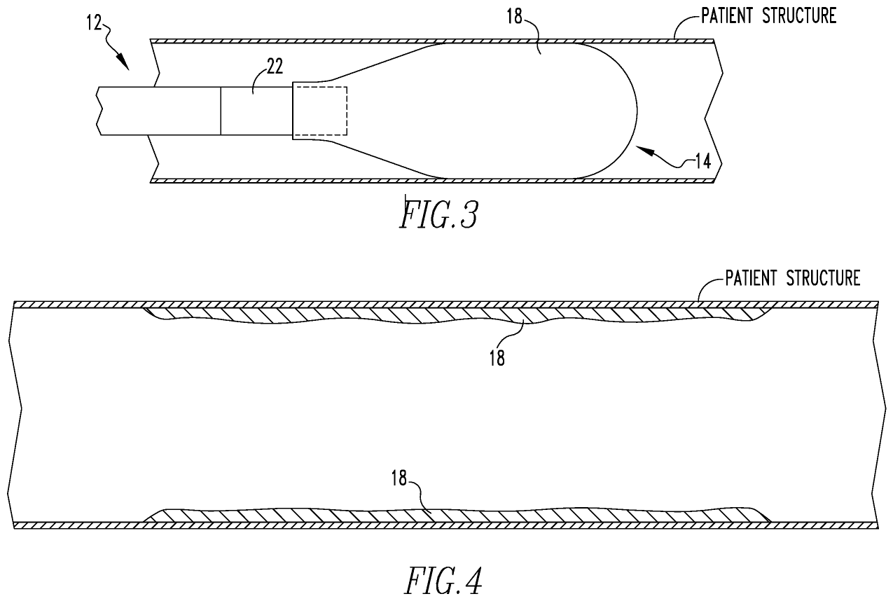

The present invention pertains to a method for treating a patient. The method comprises the steps of inserting a deflated balloon 14 having an outer surface 16 coated with a contrast medium 18 with a catheter 12 in the patient. There is the step of positioning the deflated balloon 14 with the catheter 12 to a desired position inside a vein or artery or other structure of the patient. There is the step of inflating the balloon 14 so the balloon 14 contacts and presses against an interior wall of the vein or artery or other anatomic cardiovascular structures of the patient, as shown in FIG. 3. There is the step of depositing the contrast medium 18 coating the outer surface 16 of the balloon 14 on the interior wall of the vein or artery or other anatomic cardiovascular structures. There is the step of deflating the balloon 14. There is the step of removing the balloon 14 with the catheter 12 from the patient with the contrast medium remaining on the patient structure, as shown in FIG. 4. There is the step of positioning properly an object in the patient at the desired location by comparing a position of the object with the contrast medium 18 deposited on the interior wall of the vein or artery or other anatomic cardiovascular structures.

In the operation of the invention, there is an apparatus 10 comprising a catheter 12 with a non-compliant, compliant or ultra-compliant balloon 14 disposed on the catheter 12, as shown in FIG. 1, which may be used for treating a patient. The balloon 14 includes a contrast medium 18 coating covering the outer surface 16 of the balloon 14, as shown in FIG. 2. The apparatus 10 may be used for treating a cardiovascular condition, or other conditions in a patient where it is desired to mark a desired location of the patient itself. The contrast medium 18 coating covering the outer surface 16 of the balloon 14 is deposited onto the structure of interest of the patient, such as a vein or artery, when the balloon 14 is at a desired structure location in the patient, and the balloon 14 is inflated. When the balloon 14 is inflated, the outer surface 16 of the balloon 14 makes contact with the interior wall of the structure, so the contrast medium 18 coating on the outer surface 16 of the balloon 14 also contacts the interior wall and transfers to the interior wall, as shown in FIG. 3. The mode of transfer of the contrast medium 18 from the outer surface 16 of the balloon 14 can be by rubbing off of the outer surface 16 onto the interior wall or sticking to the interior wall which the contrast medium 18 contacts.

The balloon 14 is placed and inflated in the patient in the same way that a balloon 14 with a drug coating is placed and inflated in n patient. The balloon 14 attached to the catheter 12 is inserted in the patient until the balloon 14 readies the desired positioned in the patient Contrast medium 18 flows into the balloon 14 through the catheter 12, as is well known in the art, under pressure to inflate the balloon 14 until the outer surface 16 of balloon 14 with the contrast medium 18 contacts and presses against the inner wall of the vein or artery. By the outer surface 16 of the balloon 14 pressing against the inner wall of the vein or artery, it increases the amount of contrast medium 18 on the outer surface 16 of the balloon 14 which sticks to or rubs off onto the inner wall of the vein or artery. The balloon 14, in the inflated state, presses against the inner wall of the vein or artery so the inner wall is slightly deformed by inflated balloon 14, but not some much that the structural integrity of the vein or artery is in any way compromised, as is well known in the art. The contrast medium 18 used to inflate the balloon 14 is either Iopamidol, Iohexol, ioversol or ioxaglate. When the balloon 14 is removed, the contrast medium 18 remains on the inner wall of the anatomic structure of the patient to mark the location, as shown in FIG. 4. The contrast medium 18 on the inner wall of the anatomic structure should remain for more than 30 minutes so the anatomic structure stays marked and visible from the medium 13 for subsequent procedures.

To manufacture the balloon 14 with the contrast medium 18 on the outer surface 16, a conventional catheter 12 balloon 14 (non-compliant, compliant or ultra-compliant) with diameter size ranging from 3 mm (typically for sizing vessels) to 40 mm (for larger vessels or intra-cardiac structures) and length from 15 mm to 80 mm is used. Balloons 14 will be inflated prior to coating. A balloon 14 catheter 12 coating system is used (Sonotek or other ultrasound spraying system). A rotational motor rotates the catheter 12 and balloon 14 360 degrees while a precision clamping device securely holds the catheter 12 tail in place. Iopamidol is n nonionic, low-osmolar iodinated contrast agent, used during cardiac catheterization and Computed Tomography. A highly concentrated solution of at least 370 mgI/mL is used to coat the balloon 14. When dried, the catheter 12 balloon 14 will be deflated and packed. Other contrast agents that may be used are Iohexol, ioversol or ioxaglate. An example of a balloon 14 that may be used is 3n AGA TM sizing balloon, available from AGA Medical Corporation. 682 Mendelssohn Avenue, Golden Valley, Minn. 55427. Other standard sizing balloons available in the marketplace may also be used.

Another aspect of the present invention provides a method of treating a cardiovascular condition using a contrast medium 18 coated balloon 14. The method includes the steps of delivering a balloon 14 with contrast medium 18 coating to a target region of an anatomic structure via a catheter 12 and dissolving the contrast medium 18 coating to the target region of the anatomic structure while the balloon 14 is delivered to the target region to mark the target region.

The foregoing and other features and advantages of the invention will become further apparent from the following detailed description of the presently preferred embodiments, read in conjunction with the accompanying drawings. The detailed description and drawings are merely illustrative of the invention, rather than limiting the scope of the invention being defined by the appended claims and equivalents thereof.

Applications.
a) Use of contrast-coated balloon (CCB) to mark coronary arteries before stent placement. In certain occasions the location of stent placement is of significant importance. Traditionally, a reference of u previous angiogram is used. The CCB can mark the exact location of the artery that needs to be stented. For example, marking the ostium of the left main artery and the ostium of the right coronary artery can allow accurate stent placement without unnecessary stent extention to the aorta or “missing” the coronary ostium. In cases of bifurcation stenting, CCB can mark the location of the side blanch in ease re-wiring is needed. In cases of CTO interventions, CCB can mark the distal vessel, decreasing the need for repeated coronary injections.
b) Use of contrast-coated balloon (CCB) to mark peripheral arteries before stent placement. CCB can mark “covered” vessels before covered stent placement (i.e. renal arteries with AAA endograft) and allow possible puncture through the covered stent to regain access.
c) Use of contrast-coated balloon (CCB) to mark the aortic valve before TAVR. The inaccurate placement of a transcatheter aortic valve replacement (TAVR) prosthesis has been associated with para valvular leak (PVL), subaortic obstruction and increased need for permanent pacemaker placement. Traditionally, an aortogram is used to guide placement. The use of CCB to mark the aortic valve before TAVR placement, allows the perfect placement of the TAVR prosthesis by continuous real-time visualization of the aortic valve and aortic leaflets.
d) Use or contrast-coated balloon (CCB) to mark the mitral valve before MitraClip placement of TMVR. Traditionally, the mitra-clip placement is guided by TEE. However, visualization of the leaflets is often suboptimal. Hie use of CCB before MitraClip allows the continuous real-time visualization of the mitral valve leaflets to assure capture of the leaflets with the MitraClip. Moreover, it allows perfect alignment of TMVR. The same applies for Tricuspid valve and pulmonic valve structural interventions.

Use of contrast-coated balloon (CCB) to mark the left atrial appendage (LAA) before placement of LAA occlusion device (i.e. Watchman device). Accurate LAAO device placement is necessary to prevent device embolization and complete seal of the LAA. CCB marks the LAA to guide perfect device placement
f) Use of contrast-coated balloon (CCB) to mark PDA and ASD/PFO closure procedures. CCB can mark the “tunnel” of the ASD/PFO or PDA, to guide accurate closure device placement.
g) Use of contrast-coated balloon (CCB) to assist transeptal puncture. Transeptal puncture is a complex and potentially life-threatening procedure, mainly due to the complex anatomy and the need for 2-Dimention imaging modalities (TEE or ICE) CCB can mark the right atrium and the intra-atrial septum or also the aortic route to allow accurate puncture
h) Use of contrast-coated balloon (CCB) to assist the trasncaval access. Transcaval access is a novel access technique for TAVR placement. CCB can mark the aortic wall an allow puncture from the inferior vena cava to the aorta.

Although the invention has been described in detail in the foregoing embodiments for the purpose of illustration, it is to be understood that such detail is solely for that purpose and that variations can be mace therein by those skilled in the art without departing from the spirit and scope of the invention except as it may be described by the following claims.

1. An apparatus for treating a patient comprising:
a catheter; and
a balloon having an outer surface disposed on the catheter, the balloon includes a contrast medium disposed on and covering the outer surface of the balloon, the balloon and catheter adapted to be positioned in the patient the balloon having an inflated state where the balloon is expanded in size and an uninflated state, where the balloon is unexpanded.

2. The apparatus of claim 1 wherein the contrast medium includes either Iopamidol, Iohexol, ioversol or ioxaglate.

3. The apparatus of claim 2 wherein the balloon has a diameter size ranging from 3 mm to 40 mm and u length from 15 mm to 80 mm.

4. The apparatus of claim 3 wherein the catheter is hollow and has an input port through which pressurized fluid flows to the balloon, and an output port on which the balloon is disposed to receive the pressurized fluid to inflate the balloon into the inflated state.

5. The apparatus of claim 4 wherein the pressurized fluid is also a contrast medium that includes either Iopamidol, Iohexol, Ioversol or ioxaglate.

6. A method of making a balloon with a contrast medium of an outer surface of the balloon comprising the steps of:
inflating the balloon;
placing the contrast medium on the outer surface of the balloon;
drying the contrast medium of the outer surface of the balloon; and
deflating the balloon.

7. The method of claim 6 wherein the placing step includes the steps of rotating the balloon and spraying the contrast medium onto the outer surface of the balloon.

8. The method of claim 7 including the step of clamping the balloon.

9. A method for treating a patient comprising the steps of:
inserting a deflated balloon having an outer surface coated with u contrast medium with a catheter in the patient;
positioning the deflated balloon with the catheter to a desired position inside a vein or artery of the patient;
inflating the balloon so the balloon contacts and presses against an interior wall of the vein or artery or other anatomic cardiovascular structures of the patient;
depositing the contrast medium coating the outer surface of the balloon on the interior wall of the win or artery or other anatomic cardiovascular structures;
deflating the balloon;
removing the balloon with the catheter from the patient; and
positioning properly on object in the patient at the desired location by comparing a position of the object with the contrast medium deposited on the interior wall of the vein or artery or other anatomic cardiovascular structures.
